(19) United States
(12) Patent Application Publication
(10) Pub. No.: US 2004/0043394 A1
(43) Pub. Date: Mar. 4, 2004

(54) Gene expression element specific for Ah receptor ligands and heterologous gene expression systems dependent on the element

(76) Inventor: Hideo Ohkawa, Kobe-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/375,269

(22) Filed: Mar. 3, 2003

(30) Foreign Application Priority Data
Aug. 30, 2002 (JP) 2002-254640

(51) Int. Cl. C12Q 1/68; C07H 21/04; C07K 14/705; C12N 15/00

(52) U.S. Cl. 435/6; 435/69.1; 435/320.1; 435/325; 530/350; 536/23.5

(57) ABSTRACT

There is provided a vector comprising a first promoter sequence which constitutively or conditionally regulates transcription, a first transcription structure including a DNA-binding region, a nucleus localization signal sequence, an AhR-ligand binding control region and a transcriptional activation region, which is arranged downstream of the first promoter sequence, one or more second promoter sequence which is specifically coupled with the DNA-binding region, thereby to transcribe a transcription unit under control of the transcriptional activation region, and a second transcription structure including a reporter gene, which is arranged downstream of the second promoter sequence. Also, there are provided a transformant with the vector, and a method for monitoring and/or reducing the AhR-ligand.

### CROSS-REFERENCE TO RELATED APPLICATIONS

This application is based upon and claims the benefit of priority from the prior Japanese Patent Application No. 2002-254640, filed Aug. 30, 2002, the entire contents of which are incorporated herein by reference.

### BACKGROUND OF THE INVENTION

1. Field of the Invention

The present invention relates to a novel gene expression element which specifically responds to a variety of ligands of an arylhydrocarbon receptor (AhR), and a heterologous gene expression system dependent on the element. Further, the present invention relates to techniques of monitoring AhR-ligands such as dioxins (HAHs; halogenated aromatic hydrocarbons) and/or polycyclic aromatic hydrocarbons (PAHs), and reducing the ligands from environment, by using a eukaryote in which a heterologous gene expression system have been introduced. Said system is dependent on the function of the element of this invention.

2. Description of the Related Art

At present, with development of human life and industrial activity, variety of chemicals are released to the environment, and this becomes an environmental pollution. Among these chemicals, “Chemicals for Environmental Load” (referred hereinafter as CELs) are stable, and therefore, remain in the environment for a long time. The CELs are such as dioxins, polycyclic aromatic hydrocarbons, endocrine disrupting chemicals (environmental hormones) and pesticide residues and the like. These CELs are detected in the atmosphere, water, soil and agricultural products at a level of ppt, ppb or ppm, and are known to have an influence on the ecosystem at an extremely low concentration. Therefore, a techniques of monitoring distribution and movement of CELs in the environment, and/or a techniques of reducing the CELs from the environment, are demanded.

Monitoring of the CELs by the prior art have been performed by collecting samples at many places to be monitored, transporting the samples to an experimental facility, and detecting and quantifying the CELs using instrumental analysis. Instrumental analysis is excellent in its sensitivity and precision, but it requires facilities and skilled techniques and also it takes time. Also the cost of apparatus, reagent and the like is high. Therefore, a simple, rapid, highly sensitive and inexpensive method has been demanded.

The CELs are recognized and metabolized by organisms. The metabolized chemicals are then excreted from the organisms. The monitoring techniques based on such biological function of organisms would easily be publicly acknowledged. Since risks, such as secondary pollution due to frequent use of organic solvents, are minimal.

Previously, several methods of monitoring CELs by utilizing the biological function have been developed. There are several methods for assaying environmental hormones using the receptors involved in the endocrine system (such as, female hormone like estrogen, male hormone like androgen, and thyroid hormone). These methods utilize the responsiveness of the receptors to the environmental hormones. In addition, there is also known an immunochemical method specific for CELs utilizing the specificity between antigen and antibody.

Among the CELs, “dioxins” which is a general term of substances such as polydibenzo-para-dioxin chloride (PCDDs), polydibenzofuran chloride (PCDFs) and coplanar-PCB (Co-PCBs) and “polycyclic aromatic hydrocarbons” such as benzpyrene and methylcholanthrene exhibit a variety of biological toxicities such as immunotoxicity, teratogenicity and carcinogenesis promotion in mammals. Therefore the dioxins and polycyclic aromatic hydrocarbons are especially harmful for the ecosystem and human health. For dioxins, a kit for in vitro detection using a receptor system is commercially available.

However, the prior art may exhibit an excessive reaction due to non-specific adsorption in some cases.

In addition, the prior art requires transporting samples to a laboratory from collecting point one after another, and requires reagents and apparatuses suitable for analysis. Therefore, the prior art cannot be practiced in the places where there are no experimental facilities or where it is substantially difficult to utilize a facility (e.g., field remote from experimental facility).

Further, the prior art may obtain result of measurement only after analysis in the experimental facility. Therefore rapid on-site measurement is impossible.

On the other hand, for reducing the CELs, physicochemical or biological method (e.g., using microorganisms) has been tried. However, it is extremely difficult to reduce the substances, which present widely in water, soil, atmosphere and agricultural products with extremely low concentration (e.g., the CELs), with the prior art. Consequently, a novel technique will be required to deal with the effective reduction of such substances.

There are many problems (as mentioned above) to be solved in the prior art.

An object of the present invention is to provide a vector using a novel gene expression element specifically recognizing the AhR-ligand. Another object of the present invention is to provide a transformant by developing a heterologous gene expression system, more specifically by transforming with the vector. Still another object of the present invention is to provide a method of monitoring the CELs in an environment using a eukaryote in which the heterologous gene expression system is introduced, and a method of reducing the CELs from the environment.

### BRIEF SUMMARY OF THE INVENTION

The present invention attains an object of the present invention by means described below.

According to an aspect of the present invention, there is provided a vector comprising:

a first promoter sequence which constitutively or conditionally regulates transcription;

a first transcription structure including a DNA-binding region, a nucleus localization signal sequence, an AhR-ligand binding control region and a transcriptional activation region, which is arranged downstream of the first promoter sequence;

one or more second promoter sequence which is specifically coupled with the DNA-binding region, thereby to transcribe a transcription unit under control thereof; and

a second transcription structure including a reporter gene, which is arranged downstream of the second promoter sequence;

wherein the first promoter sequence and the first transcription structure form a first set, the second promoter sequence and the second transcription structure form a second set, and the first and the second sets are arranged in cis- or trans-position on a chromosome or an episome resided within a eukaryotic cell; and

wherein an AhR-ligand which has invaded in the cell binds to a translated product of the first transcription structure, and forms a complex to an amount depending on the AhR-ligand, thereby enhancing transcription of the second transcription structure depending on the amount of the complex formed.

According to another aspect of the present invention, there is provided a transformant transformed with the vector of the invention.

According to still another aspect of the present invention, there is provided a method of monitoring an AhR-ligand, comprising a step of culturing or cultivating the above transformant, wherein the existence of the AhR-ligand in the growth environment of the transformant is monitored by expression of the reporter gene in the transformant.

According to still another aspect of the present invention, there is provided a method of reducing an AhR-ligand present in a growth environment of the above transformant, from the growth environment, comprising a step of culturing or cultivating the transformant, wherein the AhR-ligand is metabolized in a living body of the transformant.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 is a view showing a structure and a function of an arylhydrocarbon receptor (AhR).

FIG. 2A is a view showing a structure of a novel AhR-ligand-specific gene expression element (XDV/XVD).

FIG. 2B is a view showing a structure of a novel AhR-ligand-specific gene expression element (XDV/XVD).

FIG. 2C is a view showing a structure of a novel AhR-ligand-specific gene expression element (XDV/XVD).

FIG. 2D is a view showing a structure of a chimeric AhR (AhRV).

FIG. 3 is a view showing an outline of a method of assessing a performance using a yeast reporter assay system for XDV/XVD. Yeasts introduced with various XDV/XVD expression plasmids were pre-cultured overnight in a liquid selective medium containing glucose as a carbon source, cultured for 14 to 16 hours in a liquid selective medium containing an AhR-ligand and galactose(as a carbon source), and the LacZ activity was measured.

FIG. 4 is a view showing a method of producing a monitoring plant in which a gene expression system utilizing a function of AhR is introduced (the plant expresses a reporter gene in response to the existence of an AhR-ligand), and a plant for reducing pollution which expresses a drug metabolizing enzyme gene, as well as an outline of action and mechanism of the method.

FIG. 5 is a graph showing an activity of a LacZ gene which is specifically expressed by the increased concentration of AhR-ligands within the yeast introduced with XDV/XVD.

FIG. 6 is a graph showing an activity of a LacZ gene which is specifically expressed by the treatment with various AhR-ligands within the yeast introduced with XDV/XVD.

FIG. 7 is a view showing a plant expression plasmid having a GUS gene inducing expression system utilizing a function of AhR.

FIG. 8 is a photograph of electrophoresis showing RT-PCR analysis on a transformed tobacco plant.

FIG. 9 is a graph showing a GUS-activity expressed in a transformed tobacco plant. The GUS expression was induced by treating the plant with 20-methylcholanthrene.

FIG. 10 is a graph showing a GUS-activity expressed in a transformed tobacco plant. The GUS expression was induced by treating the plant with various AhR-ligands.

FIG. 11 is a graph showing a GUS-activity expressed in a transformed tobacco plant. The GUS expression was induced dose dependently by treating the plant with an AhR-ligand (20-MC).

FIG. 12 is a photograph showing a GUS activity of 20-MC-treated transformed tobacco plant in the tissue.

### DETAILED DESCRIPTION OF THE INVENTION

The present invention will be explained in detail.

Among AhR-ligands, dioxins has a toxicity, even at an extremely low concentration, especially to mammals. Thereupon, dioxins specifically bind to an arylhydrocarbon receptor (AhR) within a cell. Thereafter, a receptor complex with a ligand forms a heterodimer with an AhR nucleus transferring protein (Arnt). Then, this heterodimer induces a CYP1A1 gene as a result of specific binding to an expression control region (i.e. XRE sequence) residing 5′ upstreme of a CYP1A1 gene. As a result, transcription of a target gene such as a CYP1A1 gene is activated.

Although the CYP1A1 produced via this transcription metabolizes certain dioxins, it does not metabolize 2,3,7,8-TCDD having the highest toxicity. The extent of the toxicity of a dioxin isomer is determined based on toxicity equivalent factor (TEF) which is a relative toxicity coefficient letting the toxicity of strongest toxic 2,3,7,8-TCDD to be 1, by comprehensively assessing influence on various living bodies and in vitro experimental data in an animal experiment. When cultured mammal cell is used, a signal transduction system via AhR is capable to detect 0.01 ppb TCDD (while an environmental standard value of dioxins is 1 ppb and an examination index value therefor is 0.25 ppb).

As described, a mammalian AhR can lead to a transcription of some genes of a particular enzyme system after recognition of an extremely low concentration of AhR-ligand (e.g. dioxins).

Accordingly, the inventor gained an idea that the above-mentioned biological phenomenon in respect to AhR can be utilized for monitoring of CELs and/or reduction of pollution due to the chemicals. Then, the inventor have achieved the present invention.

The present invention utilizes the function of AhR, more specifically the function that the mammalian AhR can recognize extremely low concentration of AhR-ligand.

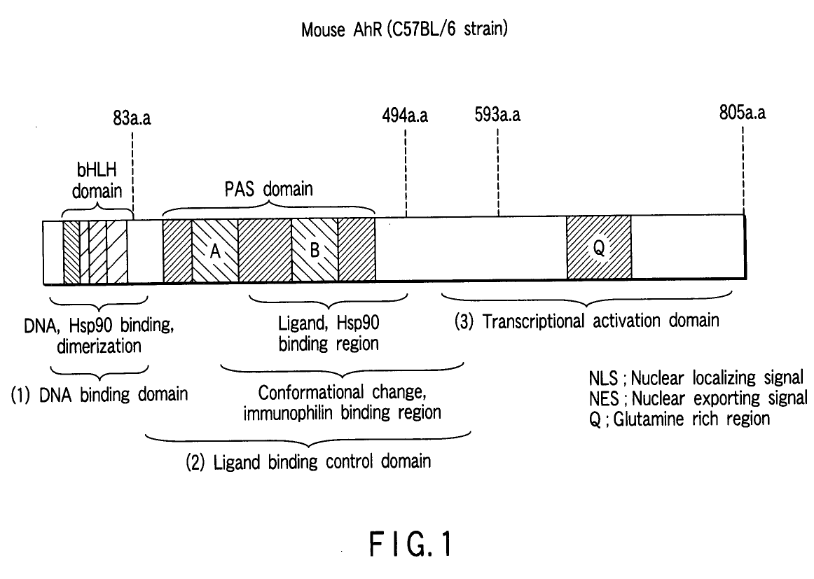

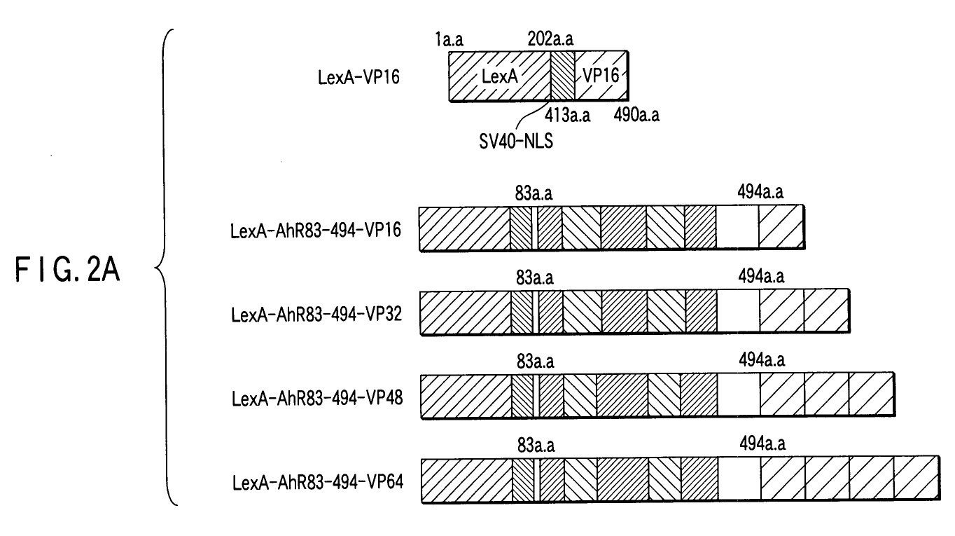

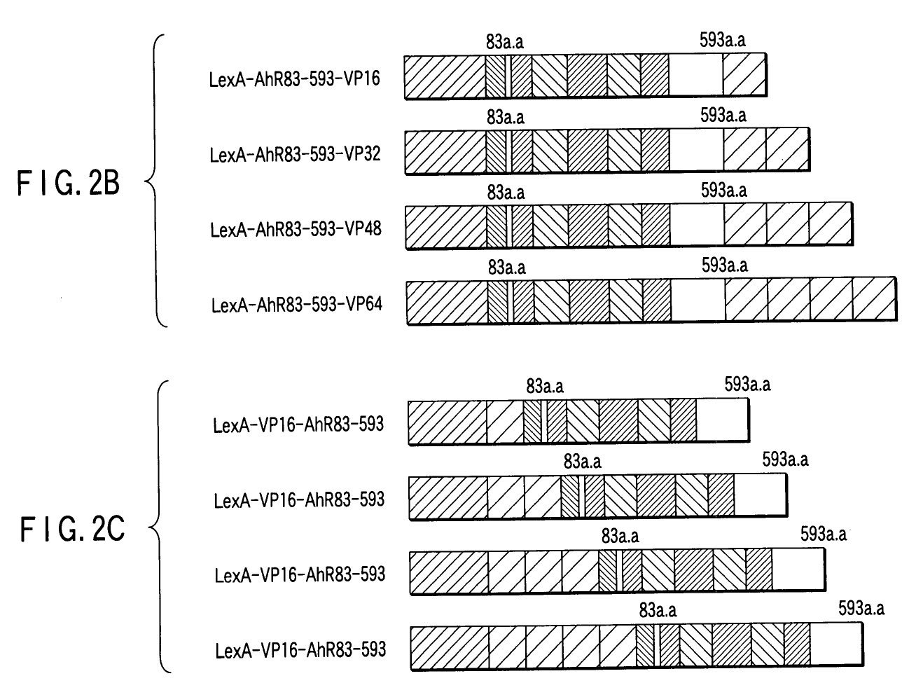

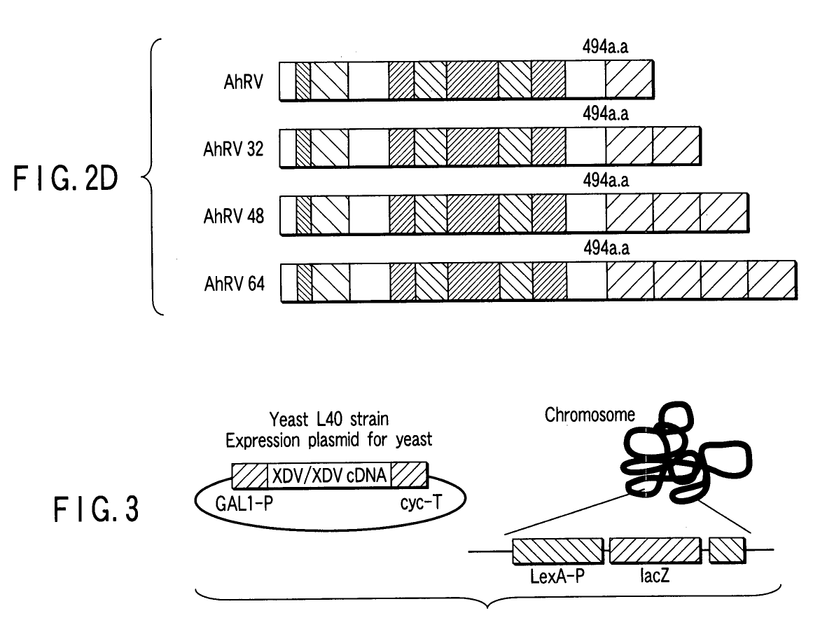

As shown in FIG. 1, AhR domain structure can be divided into three functional regions. Each region encodes one domain: (1) a DNA-binding domain, (2) a ligand binding control domain, and (3) a transcriptional activation domain. Among these regions, an element of the invention comprises a ligand binding control region(namely, this region encodes the ligand binding control domain) as an essential part of the element. By fusing homologous or heterologous functional regions such as a DNA-binding region and a transcriptional activation region of herpes virus VP16 factor suitable for essential part, a novel gene expression element was generated. This element is considerably sensitive to and specific for AhR-ligands (FIG. 2A to FIG. 2D).

In addition, the present invention utilizes a simplified system which does not require any additional factors such as Arnt. The Arnt is required for initiation of native AhR function (FIG. 3).

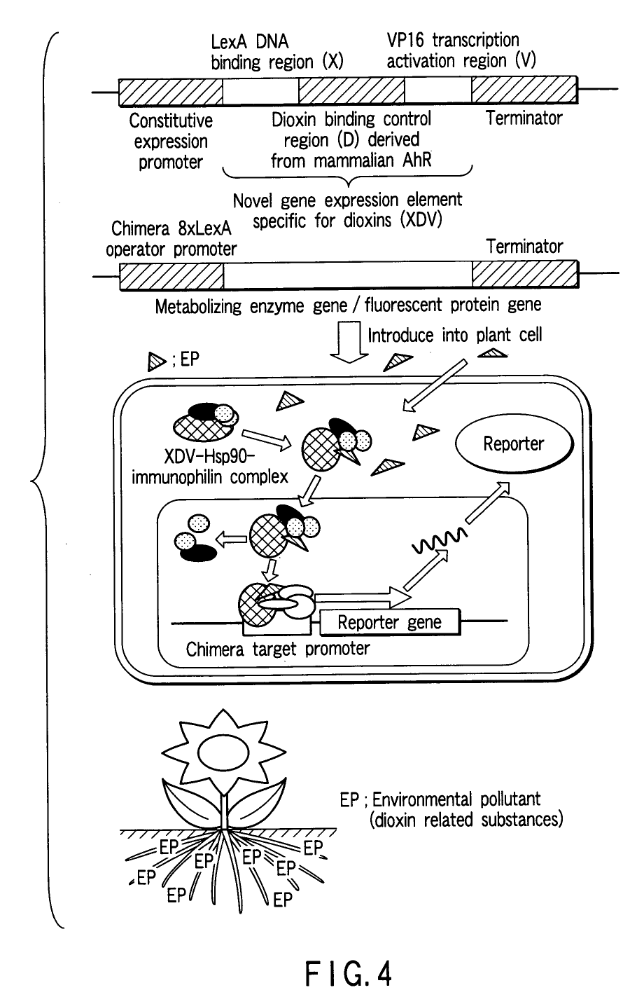

Previously, there is no report on a gene expression element of this kind, namely, the element utilizes the mechanism regulated by AhR-ligand binding. Therefore, the present invention demonstrates an AhR-ligand-specific heterologous gene expression system introduced into a plant and yeast (FIG. 4).

The present invention will be explained more specifically below.

The present invention attains the object of the present invention by using a transformant in which a vector described below is introduced. A vector of the present invention comprises:

a first promoter sequence which constitutively or conditionally regulates transcription;

a first transcription structure including a DNA-binding region, a nucleus localization signal sequence (NLS), an AhR-ligand binding control region and a transcriptional activation region, which is arranged downstream of the first promoter sequence;

one or more second promoter sequence which is specifically coupled with the DNA-binding region, thereby to transcribe a transcription unit under control thereof; and

a second transcription structure including a reporter gene, which is arranged downstream of the second promoter sequence,

wherein the first promoter sequence and the first transcription structure form a first set, the second promoter sequence and the second transcription structure form a second set, and the first and the second sets are arranged in cis- or trans-position on a chromosome or an episome resided within a eukaryotic cell; and

wherein an AhR-ligand which has invaded in the cell binds to a translated product of the first transcription structure, and forms a complex to an amount depending on the AhR-ligand, thereby enhancing transcription of the second transcription structure depending on the amount of the complex formed.

In the vector defined above,

the first promoter sequence may be any promoter which constitutively or conditionally regulates an expression of the first transcription structure, and should not be limited to any particular promoter sequence.

The term “constitutively regulates the expression” means the situation that the promoter regulating the expression is constantly active in said cell. The first promoter sequence which “constitutively regulates the expression” may be a constitutive promoter generally used in the prior art, preferably a CaMV35S (cauliflower mosaic virus 35S) promoter sequence or a G-box promoter sequence in the case of plant.

The term “conditionally regulates the expression” means the situation that the expression substantially occurs only at an intended time in the vector transfected cell. The first promoter sequence which “conditionally regulates the expression” may be a promoter generally used in prior art for inducing a gene only at a intended time, preferably a GAL1 promoter in the case of yeast. In this case, by adding galactose to a medium, the expression under control of the first promoter sequence can be induced.

A person skilled in the art can select a suitable first promoter sequence depending on the purpose and/or a subject to be transformed.

The DNA-binding region is a region which specifically binds to the second promoter sequence, and is appropriately selected by a combination with the second promoter sequence. For example, if the DNA-binding region is a DNA-binding region of AhR, it is preferable to select one or more XRE sequences for the second promoter sequence. This “XRE”, xenobiotic responsive element, is a cis-acting transcription controlling sequence which respond to a xenobiotic such as an AhR-ligand. In addition, “XRE” is the substantially synonymous with dioxin responsive element (DRE) which is a cis-acting transcription controlling sequence responding to dioxins.

A person skilled in the art can select a suitable “XRE” sequence with reference to the known publication (for example, Amy Lusska, et al., 1993).

The DNA-binding region may be a DNA-binding region of AhR, more specifically such as a region corresponding to amino acid Nos. 1 to 82 residues of mouse AhR. When a DNA-binding region of mouse AhR is used as the DNA-binding region, a complex formation of the AhR with an Arnt is required for enhancing transcription activation.

The DNA-binding region may also be a DNA-binding region of LexA, more specifically such as a region corresponding to amino acid Nos. 1 to 202 residues of bacterial repressor LexA. In this case, it is preferable that one or more LexA promoter sequences are selected for the second promoter sequence.

The second promoter sequence of the invention may be a promoter sequence, preferably a plurality of promoter sequence in which a plurality of the unit consisted of one promoter sequence are tandemly arranged.

A person skilled in the art can select a suitable combination of the DNA-binding region and the second promoter sequence.

The nucleus localization signal sequence of the invention may be any nucleus localization signal sequence which is capable to be transferred to a nucleus, more specifically a signal sequence which a translated product of the first transcription structure including said signal sequence itself is capable to be transferred to a nucleus, and should not be limited any particular type of signal sequence. Preferably, an SV40-derived nuclear localization signal sequence is used.

The AhR-ligand binding control region of the invention may be any region encoding an AhR-ligand binding control domain of AhR, and should not be limited to any particular origin. More specifically, the region is a region encoding an AhR-ligand binding control domain of mammalian (such as human, rat and guinea pig) origin, preferably of mouse origin, more preferably of a range corresponding to amino acid Nos. 83 to 593 of mouse origin, most preferably of a range corresponding to amino acid Nos. 83 to 494 of mouse origin. In other words, the AhR-ligand binding control region may be a region fulfills expected function of the first transcription structure containing said region. The expected function is, to bind specifically to an AhR-ligand to form a complex, thereby enhancing transcription of the second transcription structure as a result.

The transcriptional activation region of the invention may be any region encoding an transcriptional activation domain, and should not be limited to any particular origin. More specifically, said region is a region encoding a transcriptional activation domain of AhR derived from various organisms, preferably of VP16 protein derived from herpes simplex virus, more preferably of a range corresponding to amino acid Nos. 413 to 490 of the VP16 protein derived from herpes simplex virus. In addition, said region may contain more than one transcriptional activation domain of VP16 protein. In this case, a plurality of said domain are tandemly aligned within the first transcription structure. In other words, the transcriptional activation region may be a region fulfills expected function of the first transcription structure containing said region. The expected function of said structure is, to bind specifically to an AhR-ligand to form a complex, thereby enhancing transcription of the second transcription structure as a result.

The term “region” in the present invention has two meanings when used regarding the vector of the present invention. That is, firstly, the word is used as a word meaning the nucleic acid having a certain chain length, and means a range of nucleic acid constituting the vector. Secondly, the word is used as a word meaning a protein, and means a whole or a domain of the protein molecule encoded by the range of nucleic acid. Therefore, each “region” means nucleic acid constituting a vector, but when the “region” is translated, it should be understood to mean a protein. In either case, the region should be recognized as a stretch of coding region correspond to a certain functional region or a domain.

In the vector of the present invention, the first transcription structure which is arranged downstream of the first promoter sequence is a transcription unit including the DNA-binding region, the nucleus localization signal sequence, the AhR-ligand binding control region and the transcriptional activation region, and is transcribed under control of the first promoter sequence. This first transcription structure is required to contain the DNA-binding region, the nucleus localization signal sequence, the AhR-ligand binding control region and the transcriptional activation region, otherwise it should not be limited to any particular form. However, it is preferable that the DNA-binding region, the nucleus localization signal sequence, the AhR-ligand binding control region and the transcriptional activation region are arranged in this order from a side downstream of the first promoter sequence.

Preferably, the first transcription structure may include XDV/XVD, wt-AhR (wild type AhR) or AhRV (chimera AhR) as a transcription unit.

The XDV/XVD has a domain structure as shown in FIG. 2A to FIG. 2D. More specifically, the XDV/XVD is a chimera molecule comprising (a) a DNA-binding region of LexA (region including a DNA sequence corresponding to amino acid Nos. 1-202 residues of bacterial repressor LexA), (b) NLS of SV40, (c) or (c′) a mouse AhR-ligand binding control region (region including a DNA sequence corresponding to amino acid Nos. 83-494 or 83-593, respectively, of mouse AhR), and (d) a VP16 repeat in which DNA sequence unit corresponding to a region of amino acid Nos. 413-490 in a transcriptional activation region of the VP16 protein are tandemly connected so as to form 1 to 4 repeat of the unit.

Either of domain (c) or (c′) may be used in XDV/XVD, and the domains (a) to (d) of XDV/XVD may be arranged in any order. For example, in an order from near a first promoter, it may be an order of (a)→(b)→(c)→(d) (see LexA-AhR83-494-VP16, LexA-AhR83-494-VP32, LexA-AhR83-494-VP48, and LexA-AhR83-494-VP64 in FIG. 2A). In addition, it may be an order of (a)→(b)→(c′)→(d) (see LexA-AhR83-593-VP16, LexA-AhR83-593-VP32, LexA-AhR83-593-VP48, and LexA-AhR83-593-VP64 in FIG. 2B). Further, it may be an order of (a)→(d)→(b)→(c′) (see LexA-VP16-AhR83-593, LexA-VP32-AhR83-593, LexA-VP48-AhR83-593, and LexA-VP64-AhR83-593 in FIG. 2C).

As the “wt-AhR”, a wild-type AhR having a domain structure shown in FIG. 1 can be used. The wild-type AhR is a naturally occurring intact AhR molecule, and may be derived from a variety of spices such as human, rat and guinea pig, preferably from mouse.

The “AhRV” is such that only a transcriptional activation region of the wt-AhR is substituted with the VP16 repeat (see AhRV, AhRV32, AhRV48, AhRV64 in FIG. 2D). When the AhRV is mouse-derived AhR, it is preferable that a part after amino acid No. 495 of mouse AhR is substituted with the VP16 repeat.

The second promoter sequence may be any promoter sequence which is specifically coupled with the DNA-binding region, thereby to transcribe a transcription unit under control thereof, and should not be limited to any particular promoter sequence. However, as explained above, the second promoter sequence specifically coupled with the DNA-binding region. Therefore, it is preferable that the second promoter sequence is appropriately selected by a combination with the DNA-binding region.

In first embodiment of the process of the invention, a term “reporter gene” should be recognized as a gene which comply with the object of the embodiment explained above in the “method of monitoring an AhR-ligand”. Therefore, this word means the gene such that a translated product of a gene can be detected and/or quantified.

In the first embodiment, the reporter gene may be a gene encoding for example, LacZ, β-glucuronidase (GUS), green fluorescent protein (GFP) or cytochrome p450. In particular, LacZ, β-glucuronidase (GUS), green fluorescent protein (GFP) and the like are generally used reporter genes, and a person skilled in the art can easily detect and/or quantify them. Generally, a method based on an activity and a nature of a translated product of the reporter gene can be used for detection and/or quantification. In addition, those which can be detected and/or quantified using a specific antibody thereto may be also used. Therefore, the reporter gene may also be a nucleic acid encoding a protein or a fragment thereof having the known antigenicity.

In addition, reporter genes described regarding a first embodiment and a second embodiment may be optionally used by combining them. In this case, a method of expressing a plurality of proteins as a fused protein may be used.

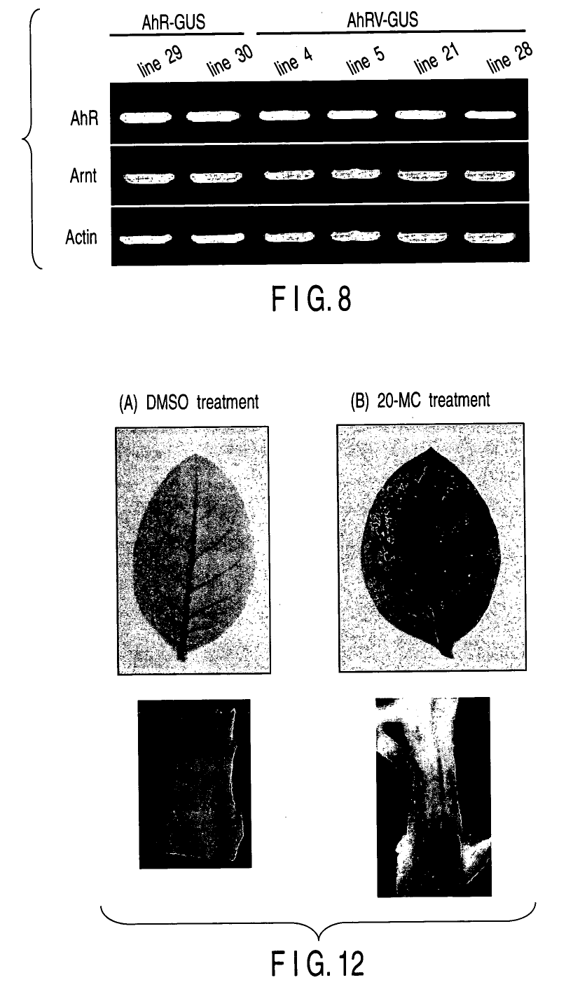

Alternatively, the first embodiment may be also performed by detecting and/or quantifying an mRNA which is a transcript of the reporter gene. A method for detecting and/or quantifying the transcript may be such as RT-PCR (see FIG. 8).

In second embodiment of the process of the invention, this word should be recognized as a gene which comply with the object of the embodiment explained above in the “method of reducing an AhR-ligand from the growing environment”. Therefore, this word also means a gene which can metabolize an AhR-ligand. In the embodiment, the reporter gene may be for example a drug metabolizing enzyme which metabolizes an AhR-ligand into a substance harmless to organisms. Preferably, the drug metabolizing enzyme may be a cytochrome p450 gene.

The cytochrome p450 is a general term of a gene family comprising many groups having different substrate specificities and reactions involving, and the cytochrome p450 metabolizes foreign matters and drugs which have entered into a living body and detoxicates them. A person skilled in the art can appropriately select a cytochrome p450 gene suitable for a AhR-ligand. If an AhR-ligand is for example dioxins, a CYP1A1 (CYP1A2) gene can be used for metabolizing. The gene oxidatively metabolizes the dioxins, and converts them into metabolites which are harmless to the environment, easily excreted out of a living body and are low toxic (T. Sakaki et al, 2002).

Further, the cytochrome p450 gene may be also used as the reporter gene in the first embodiment. This is accomplished by, expressing a cytochrome p450 gene which catalysts a particular reaction, contacting the produced cytochrome p450 with a substrate to cause the particular reaction in a living body, and detecting a signal, such as color development, caused by a reaction. For example, using a β-glucuronidase (GUS) gene as the reporter gene, 5-bromo-4-chloro-3-indolyl-(-D-glucuronide(X-Gluc) which is introduced as the above-mentioned particular substance is de-esterified. This reaction produces an indoxyl derivative monomer, and this substance is oxidized and polymerized with the air to produce an indigotin pigment. Then this pigment exhibits blue, whereby, monitoring of the first embodiment can be performed.

The second transcription structure is a transcription unit which is transcribed under control of the second promoter sequence, and includes one or more afore-mentioned reporter genes.

The first promoter sequence and the first transcription structure form a first set, as well as the second promoter sequence and the second transcription structure form a second set. The first set and the second set are arranged in the same molecule (cis), or are arranged separately in different molecules (trans). The molecule may be a chromosome or an episome in a cell of a eukaryote. In other words, the first set and the second set should be present in the same cell.

In addition, the first transcription structure and the second transcription structure may include a un-translated region on its 5′ side in order to improve the efficacy of translation (translation from a mRNA into a protein). An alfalfa mosaic virus or tomato mosaic virus 5′-un-translated region may be used in a plant as the un-translated region.

Also, the first transcription structure and the second transcription structure include a transcription terminating sequence suitable in an organism to be transformed, in its 3′-un-translated region. When the organism is a plant, nopaline synthase terminator (Nos-T), pea rbcS-3A terminator (T3A), and pea rbcS-E9 terminator (TE9) and the like can be used as the transcription terminating sequence.

The AhR-ligand is the substances which binds to a translated product of the first transcription structure, and forms a complex in an amount depending on an amount of the AhR-ligand, thereby enhancing a transcription of the second transcription structure depending on an amount of the complex formed. The AhR-ligand may preferably be the dioxins such as dibenzo-para-dioxin chloride (PCDDs), polydibenzofuran chloride (PCDFs) and coplanar-PCB (Co-PCBs), and/or polycyclic aromatic hydrocarbons such as benzpyrene and methylcholanthrene.

The action of a vector provided by the present invention is characterized in that, in a cell with the vector introduced therein, an AhR-ligand which has invaded in the cell binds to a translated product of the first transcription structure, and forms a complex at an amount depending on an amount of the existing AhR-ligand, whereby, transcription of the second transcription structure is enhanced depending on an amount of the complex formed. This means that the first transcription structure has a main function of the “gene expression element” in the present invention. The structure acts as a kind of a transcription factor, and enhances transcription of the second transcription structure. The functional characteristic of the first transcription structure is that the binding with an AhR-ligand results in a transcriptional activation of the structure, and the transcription activating is further enhanced according to an amount of the AhR-ligand.

Alternatively, the vector of the present invention may further comprises a third set which including a third promoter sequence and a third transcription structure. The structure comprises an Arnt gene and/or a drug resistant gene. The structure is arranged downstream of the promoter sequence and is transcribed under control of the sequence. The third set may be located in the same molecule as that of the first and/or second set.

As this third promoter sequence, the same sequence as that of the first promoter sequence can be used.

The Arnt gene is not limited by an origin of organism from which it derives. Accordingly, the Arnt genes may be derived from a variety of species such as human, rat and guinea pig.

The drug resistant gene may be one which can be used for selection of a transformed cell, and a suitable drug resistant gene may be selected depending on the purpose and/or a subject to be transformed. Generally, the drug resistant gene which can select a host cell which is used in order to amplify the vector of the present invention and/or a transformant transformed with the vector is used. As the drug resistant gene, for example, NPTII (neomycin phosphotransferase II) can be used.

The third transcription structure may comprise other genes depending on the purpose.

The third transcription structure may also includes a un-translated region on its 5′ side in order to improve the efficacy of translation. The un-translated region may be an alfalfa mosaic virus or tomatoes mosaic virus 5′-un-translated region when the organism is a plant.

The third transcription structure includes a transcription terminating sequence suitable for an organism to be transformed, in its 3′-un-translated region. The transcription terminating sequence may be a nopaline synthase terminator (Nos-T), pea rbcS-3A terminator (T3A), and pea rbcS-E9 terminator (TE9) when the organism is a plant.

The above-mentioned “the third set may be located in the same molecule as that of the first and/or second set” generally means that, the third promoter sequence and the third transcription structure are contained in the same molecule as that of the first and/or a second transcription structure. In this case, the first to third transcription structures are arranged in the same vector, and as a result, the first to third transcription structures are arranged in the same molecule in a transformed cell.

However, it is only required that each promoter and each transcription structure are functionally present within the same cell of a eukaryote, and the positions of each molecules are not limited.

A person skilled in the art can suitably construct the vector of the present invention based on the known method. The sequence information for a nucleic acid encoding each region constituting the vector of the present invention can be obtained by retrieving the known sequence information from a database. In addition, the nucleic acid can be obtained by using a PCR method employing primers designed based on the above-mentioned sequence information, and a library including the nucleic acid as a template.

The library including the nucleic acid as a template, which is derived from various organs of various organisms, can be obtained commercially or from each depositary organization.

The obtained nucleic acids can be suitably ligated using the known molecular biological procedure to construct the vector of the present invention.

Also, the present invention provides a transformant transformed with the vector of the present invention.

An organism which is suitably transformed by the vector of the present invention may be a eukaryote. The eukaryote may be preferably a plant, more preferably a higher plant, most preferably tobacco can be used. Alternatively, the eukaryote may be a yeast, preferably yeast L40 strain.

When the transformant is a plant, the transformant can be produced by transforming the plant with a plant vector of the present invention. The plant vector is one aspect of the vector of the present invention, and is containing components which should be possessed by the vector of the present invention.

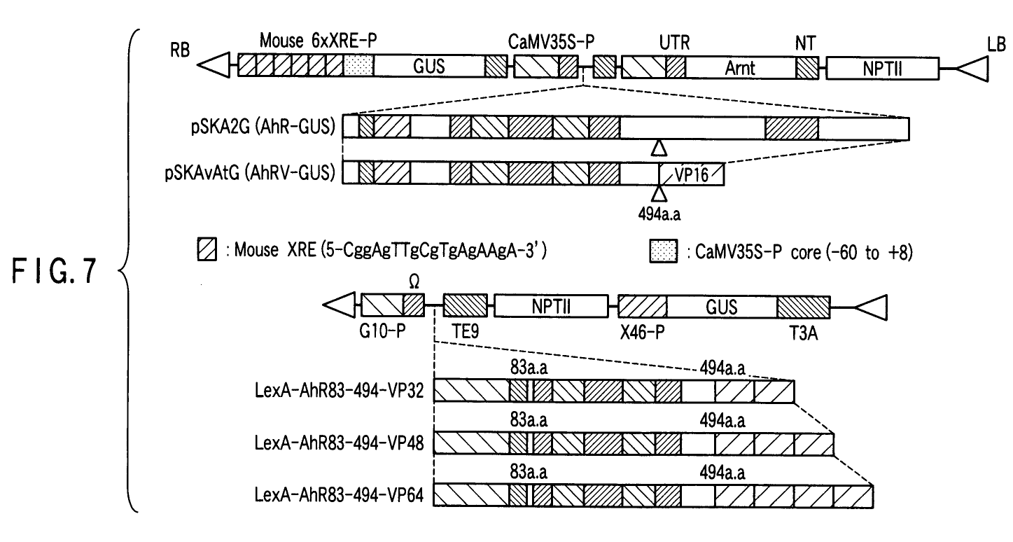

A person skilled in the art can produce a transformant by constructing the plant vector having suitable components, and for example, a vector exemplified in FIG. 7 can be used.

A vector exemplified in an upper row in FIG. 7 is a vector which is based on the T-DNA binary vector system, and is a vector composed of the followings in order from a position of RB (right border). That is, the vector comprising:

a sequence in which six mouse XRE sequences are tandemly ligated, as the second promoter sequence (continuing to CaMV35S-P core sequence);

a GUS gene, as the reporter gene included in the second transcription structure (continuing to a transcription terminating sequences of NT);

a CaMV35S promoter sequence, as the first promoter sequence;

the wild AhR or the AhRV (having an alfalfa mosaic virus 5′-un-translated region as a preceding sequence, and having a transcription terminating sequence of NT as a subsequent sequence), as the first transcription structure;

a CaMV35S promoter sequence, as a third promoter sequence;

a mouse Arnt gene (having an alfalfa mosaic virus 5′-un-translated region as a preceding sequence, and having a transcription terminating sequence of NT as a subsequent sequence) as the third transcription structure;

a nopaline synthase promoter sequence, as the third promoter sequence; and

a NPTII gene (having a nopaline synthase terminator sequence as a subsequent sequence), as the third transcription structure.

The CaMV35S-P core sequence is a sequence composed of a region which is a part of a CaMV35S promoter sequence (−60 to +8).

A vector in which the first transcription structure is the wild AhR is designated as pSKA2G (AhR-GUS), and a vector in which the first transcription structure is the AhRV is designated as pSKAvAtG (AhRV-GUS).

A vector exemplified in a lower row in FIG. 7 is also a vector which is based on the T-DNA binary vector system, and is a vector composed of the followings in an order from a position of RB (right border). That is, the vector comprising:

a G-box promoter sequence, as the first promoter sequence;

the XDV/XVD (having a tomato mosaic virus 5′-un-translated region as a preceding sequence, and having a transcription terminating sequence of TE9 as a subsequent sequence), as the first transcription structure;

a nopaline synthase promoter sequence, as the third promoter sequence;

a NPTII gene (having a nopaline synthase terminator sequence as a subsequent sequence), as the third transcription structure,

a LexA promoter sequence (expressed as X46-P in the figure), as the second promoter sequence; and

a GUS gene (having a transcription terminating sequence of T3A as a subsequent sequence), as the reporter gene included in the second transcription structure.

In addition, translations in the figures will be explained below.

AhR: Mouse AhR (derived from C57BL/6 line)

Arnt: Mouse Arnt (derived from C57BL/6 line)

CaMV35-P: Cauliflower mosaic virus 35S-promoter

GUS: β-glucuronidase

NPTII: Neomycin phosphotransferase II

UTR: Alfalfa mosaic virus 5′ un-translated region

RB: Right border

LB: Left border

NT: nopaline synthase terminator

VP16: Herpes simplex virus VP16 protein transcriptional activation domain (413-490a.a)

Ω: Tomato mosaic virus 5′ un-translated region

X46-P: LexA promoter sequence

G10-P: Chimera G-box promoter

T3A: Pea rbcS-3A terminator

TE9: Pea rbcS-E9 terminator

Using Agrobacterium with the plant vector introduced therein, this is introduced into a plant by a leaf disc method to make the transformant of the present invention.

Also in the case where the plant is tobacco, a transformant can be made similarly.

When the transformant is yeast, a yeast can be transformed with a yeast vector of the present invention to make the transformant of the present invention. The yeast vector is one aspect of the vector of the present invention, and is containing components which should be possessed by the vector of the present invention.

A person skilled in the art can make a transformant by constructing the yeast vector having the suitable components, and for example, a vector exemplified in FIG. 3 can be used.

The vector exemplified in FIG. 3 is constructed by inserting a cDNA of the XDV/XVD as the first transcription structure into an Xho I site at a multicloning site of the yeast expression plasmid pYES3/CT (Invitrogen, Netherland) in a forward direction.

The pYES3/CT has an expression cassette composed of a GAL1 promoter as the first promoter sequence, and a CYC1 terminator as a transcription terminating sequence, and can control expression of the introduced XDV/XVD in the presence of galactose as a carbon source in yeast.

The yeast L40 strain already contains a LexA promoter sequence as the second promoter sequence and a LacZ transcription unit as the second transcription structure arranged downstream to the promoter sequence in a chromosome of the strain. Accordingly, by transforming with a suitable vector such as a vector in which the XDV/XVD is introduced in the pYES3/CT, it can be used as the transformant of the present invention (see FIG. 3).

Also, the present invention provides a method for monitoring an AhR-ligand, which comprises a step of culturing or cultivating the transformant of the present invention, wherein the existence of the AhR-ligand in the growth environment of the transformant is monitored according to the reporter gene expressed in the transformant.

When the transformant is a plant, the plant is planted in the soil of a place to be measured, then cultivated over a certain period, and thereafter, the AhR-ligand present in the environment of the place can be detected and/or quantified by a method specific for the reporter gene as explained above.

Further, the present invention provides a method for reducing the AhR-ligand from the growth environment of a transformant, which comprises a step of culturing or cultivating a transformant, wherein the AhR-ligand is metabolized within the transformant.

When the transformant is a plant, the ligand can be reduced from the environment by planting and cultivating the plant in the soil of a place to be measured. The AhR-ligand present in the environment of the place is metabolized by the drug metabolizing activity of the reporter gene, as explained above.

As the AhR-ligand which is a subject of the above-mentioned method, there can be contemplated various arylhydrocarbons which can bind to AhR, and more specifically dioxins and/or polycyclic aromatic hydrocarbons are suitable subjects.

As explained above, according to the present invention, CELs in the environment can be monitored on site, and these loading chemicals can be reduced on site from the environment. Of course, samples such as the soil and water are collected from the place to be measured, and these are brought to a laboratory remote from the place, and thereafter, monitoring in accordance with the present invention can be performed using the transformant of the present invention.

### EXAMPLES

The present invention will be explained more specifically by way of Examples. However, the technical scope of the present invention is not limited to those Examples.

### Example 1

Construction of AhR-Ligand-Specific Gene Expression Element (XDV/XVD)

<Experimental Materials>

A structure of AhR derived from the mouse C57BL/6 lineage is shown in FIG. 1 (Whitelaw, M. L., et al, 1993, and Whitlock, J. P., 1999). As explained above, a structure of AhR can be roughly separated into (1) a DNA-binding domain, (2) an AhR-ligand binding control domain, and (3) a transcriptional activation domain. Accordingly, by fusing a heterologous DNA-binding region and transcriptional activation region to an AhR-ligand binding control region, a novel AhR-ligand-specific gene expression element (XDV/XVD) has been generated.

That is, in the present invention, by combining,

the AhR-ligand-specific gene expression control region (amino acid 83 to 494 residues, and amino acid 83 to 593 residues), D; a DNA-binding region (amino acid 1 to 202 residues) of and a DNA-binding region of bacterial repressor LexA, X; and a transcriptional activation region of herpes virus VP16 (amino acid 413 to 490 residues), V,

a AhR-ligand-specific gene expression element, XDV/XVD has been constructed.

From a cDNA library prepared from a liver of mouse C57BL/6 lineage, a mouse AhR cDNA was cloned. In addition, a cDNA of each of LexA and VP16 was cloned by a PCR method using pEG202 (OriGene Technologies Inc. Rockville, Md.) and Per 1 (Zuo, J., et al 2000) as a template. An XDV/XVD cDNA was constructed by introducing a restriction enzyme Xho I recognizing site into a 5′ terminal and a restriction enzyme SalI recognizing site at a 3′ terminal of each cDNA , and fusing the sites. Other DNA sequences were obtained by insertion of a synthetic DNA linker. The structures of them are described in FIG. 2A to FIG. 2D. A cDNA of each XDV/XVD was expressed in Escherichia coli, and production of a corresponding protein was confirmed by Western analysis.

<Assessment of XDV/XVD using Yeast Reporter Assay System>

By using a reporter gene assay system using a yeast expression system, assessment of performance of XDV/XVD was performed. A cDNA of each XDV/XVD was inserted into a Xho I site of a multicloning site of yeast expression plasmid pYES3/CT (Invitrogen, Netherland) in a forward direction to construct a series of yeast expression plasmids comprising each XDV/XVD. The pYES3/CT has an expression cassette composed of a GAL1 promoter and a CYC1 terminator, and can control the expression of XDV/XVD in the presence of galactose as a carbon source in yeast.

An yeast L40 strain having a reporter unit composed of a LexA operator promoter and a β-galactosidase gene (LacZ) in a chromosome [(MATa his3Δ200 trp1-901 leu2-3112 ade2 LYS2:: (41exAop-HIS3)URA3:: (81exAop-lacZ)GAL4) (Invitrogen, Netherland)) was transformed, and assessed by the LacZ activity dependent on the expression of XDV/XVD and ligand treatment as an index. A single colony was cultured at 30° C. overnight in a 1.6 mL selective liquid medium containing a suitable amino acid and glucose as a carbon source, a 160 μL of which was added to a inducing liquid medium including a suitable amino acid, galactose and raffinose as a carbon source, and an AhR-ligand to a final amount of 1.6 mL, followed by culturing at 30° C. for 16 to 18 hours.

Various AhR-ligands (dioxins) were dissolved in DMSO (a final concentration of DMSO in a medium was adjusted at 0.1%). After culturing, the yeast collected from 200 μL of a culturered solution was suspended in a Z-buffer (60 mM Na₂HPO₄, 40 mM NaH₂PO₄, 1 mM MgSO₄, 10 mM KCl, 35 mM β-mercaptoethanol). Then, it was treated with CHCl3 and 0.1% SDS. Then, a substrate solution of ortho-nitrophenyl-β-galactopyranoside (dissolved in the Z-buffer at 4 mg/ml) was added to it, and mixed to react at 37° C. A reaction was stopped by adding 1M Na₂CO₃ at appropriate time, then centrifuged. An absorbance at 415 nm of the supernatant was measured. A LacZ unit was calculated by the following calculation equation:

LacZ unit=absorbance at 415 nm/[absorbance at 600 nm of a diluted cultured yeast solution in which 200 μL of cultured yeast solution was diluted up to 1 mL]×reaction time (min)]×1000

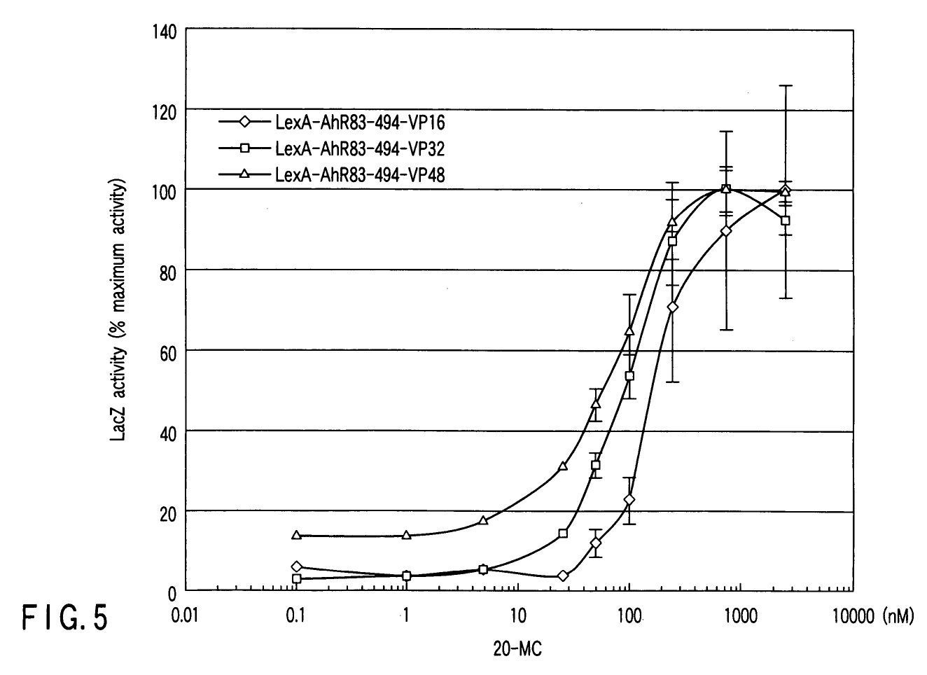

The AhR-ligands, namely, 20-methylcholanthrene (20-mc), β-naphthoflovone (β-NF) and indigo were subjected to a yeast assay system. Among XDV/XVDs subjected to an analysis, the transcription activity dependent on AhR-ligand (20-MC) treatment was recognized in 12 species of them (Table 1). Inter alia, the activities of LexA-AhR83-494-VP32, LexA-AhR83-494-VP48, LexA-AhR83-494-VP64, LexA-VP32-AhR83-593 and LexA-VP48-AhR83-593 were high, and significantly potent gene transcriptional activations specific to the treatment were found (FIG. 5).

These exhibited the equivalent or stronger activities in comparison with LexA-VP16 (XVP16) used as a positive control for assessing the performance of XDV/XVDs. The XVP16 has a very strong transcriptional activation ability. Under the similar conditions, gene expression activities of various XDV/XVDs were compared in the presence of various (7 grades) concentration of 20-MC. As a result, the concentration dependency of the transcription activity was confirmed in all XDV/XVDs. Several results is shown in FIG. 5. Inter alia, LexA-AhR83-494-VP32 and LexA-AhR83-494-VP48 showed the very high transcription activity, it was significant even at 5 nM of 20-MC.

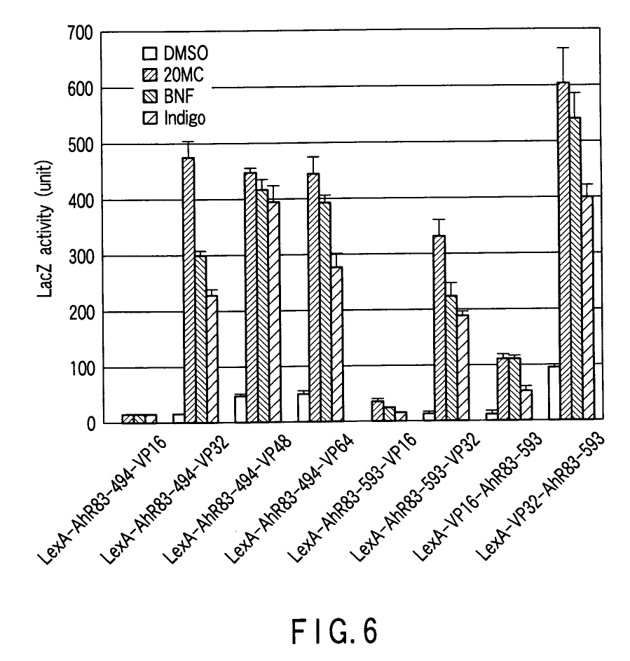

Further, under the similar conditions, the transcriptional activities of XDV/XVDs were analyzed in the presence of AhR-ligands (β-NF and indigo are other two kinds of AhR-ligands) As shown in FIG. 6, although the LacZ activities were different depending on the respective AhR-ligands, enhancement of the LacZ activity was recognized in any AhR-ligand treatment. Although not shown herein, there are only little differences regarding the LacZ activity between the condition under the presence of the 20-MC and a glucose (as a carbon source) and the condition under the presence of the DMSO and a galactose (as a carbon source). This result confirms that the LacZ activity detected in this analysis depends on the transcriptional activity of XDV/XVDs.

TABLE 1

| | Ligand Activity | |
|---|---|---|
| XDV/XVD | (unit) | |
| LexA-VP16 (Positive control) | DMSO | 404.7 ± 32.1 |
| pYES/3CT (Negative control) | DMSO | — |
| LexA-AhR83-494 | DMSO | — |
| | MC | — |
| LexA-AhR83-593 | DMSO | — |
| | MC | — |
| LexA-AhR83-805 | DMSO | — |
| | MC | — |
| LexA-AhR83-494-VP16 (4V16) | DMSO | 0.4 ± 0.1 |
| | MC | 11.3 ± 2.7 |
| LexA-AhR83-494-VP32 (4V32) | DMSO | 13.4 ± 1.9 |
| | MC | 479.0 ± 28.3 |
| LexA-AhR83-494-VP48 (4V48) | DMSO | 47.1 ± 4.4 |
| | MC | 449.0 ± 10.0 |
| LexA-AhR83-494-VP64 (4V64) | DMSO | 50.2 ± 5.0 |
| | MC | 446.3 ± 30.7 |
| LexA-V16-AhR83-494 (V1G-4) | DMSO | — |
| | MC | — |
| LexA-AhR83-593-VP16 (5V16) | DMSO | 0.3 ± 0.1 |
| | MC | 37.5 ± 2.0 |
| LexA-AhR83-593-VP32 (5V32) | DMSO | 11.0 ± 1.4 |
| | MC | 331.0 ± 32.3 |
| LexA-AhR83-593-VP48 (5V48) | DMSO | 20.7 ± 4.3 |
| | MC | 252.0 ± 52.2 |
| LexA-AhR83-593-VPG4 (5V64) | DMSO | 10.3 ± 2.1 |
| | MC | 89.1 ± 18.8 |
| LexA-VP1G-AhR83-593 (V16-5) | DMSO | 5.5 ± 0.8 |
| | MC | 110.8 ± 8.8 |
| LexA-VP32-AhR83-593 (V32-5) | DMSO | 97.0 ± 5.0 |
| | MC | 605.5 ± 61.8 |
| LexA-VP48-AhR83-593 (V48-5) | DMSO | 118.7 ± 5.4 |
| | MC | 574.8 ± 36.1 |
| LexA-VP64-AhR83-593 (V64-5) | DMSO | 100.6 ± 25.8 |
| | MC | 307.2 ± 12.9 |

### Example 2

Construction of AhR-Ligands Specific Reporter Gene Expression System using AhR

<Experimental Materials>

A AhR-ligand specific gene expression system for plants was constructed. It was constructed by introducing a AhR, Arnt and XRE to a plant. From a liver cDNA library prepared from C57BL/6 mouse, an AhR cDNA (Schmidt, J. V., et al, 1993) and an Arnt cDNA (Reisz-Porszasz, S., et al, 1994) were cloned. In order to constitutively express both cDNAs in a plant, there were constructed two kinds of plant expression plasmids pSKA2G and pSKAvAtG. The plasmids comprising a tandemly aligned XRE sequence, a transcription factor expression unit (i.e. the first transcription structure) inserted in an expression unit composed of a cauliflower mosaic virus 35S promoter (CaMV35S-P) and a nopaline synthase terminator (Nos-T), and a reporter unit composed of a reporter gene and Nos-T (structures are shown in FIG. 7). The PSKAvAtG contains a chimera AhR (AhRV) in which transcriptional activation domain of AhR is substituted with VP16 transcriptional activation domain (AhRV gene). Using Agrobacterium with each plasmid introduced therein, a tobacco plant (Nicotiana tabacum cv. Sumsun N N) was transformed by a leaf disc method.

<Experimental Method>

The plants which had been introduced with each plant expression plasmid were selected two times by antibiotic kanamycin. Each genomic DNA was extracted from re-differentiated individuals after kanamycin selection, and genomic PCR analysis was performed using primers specific for each of AhR or AhRV, Arnt and GUS. As a result, an individual, for which amplification of a cDNA band of a corresponding size was confirmed in all three kinds, was used as a transformed tobacco plant in following analysis (number of individuals is shown in Table 2).

The plant transformed with either pSKA2G or pSKAvAtG is designated as AhR-GUS or AhRV-GUS plants, respectively. Axillary buds were removed from all transformed plants. The buds were cultured for 2 to 3 week, and were grown in a medium containing an AhR-ligand (25 μM 20-MC, 25 μM β-NF, 50 μM indigo). Then the soluble protein fractions were extracted from stem and leaf parts of the grown buds, and the GUS activities of the fractions were measured. The medium were added with 0.01% Tween 20 and an AhR-ligand (20-MC) or a solvent DMSO solution to a final concentration of 0.1%. Similarly, a axillary bud was cultured for 2 weeks on an MS medium in the presence of various concentrations (8 grades) of AhR-ligand 20 MC. The AhR-ligand dose dependent expression of the reporter was analyzed. In addition, similarly, a axillary bud was cultured for 2 weeks on an MS medium containing 25 μM 20-MC, and tissue-specific expression of a reporter in a plant was analyzed using a GUS staining method. That is, each tissue of a plant was impregnation-treated in a X-Gluc solution (0.1M sodium phosphate buffer, pH 7.0, 1.9 mM X-glucuronide, 0.5 mM K₃Fe(CN)₆, 0.5 mM K₄Fe(CN)₆, 0.3% (v/v) Triton X-100, 20% methanol) under reduced pressure, then incubated at 37° C. overnight, and 70% ethanol was added to stop the reaction. Thereafter, decoloration was performed in 70% ethanol, and the tissue was observed under an optical microscope or stereomicroscope. Further, confirmation of expression of introduced genes such as AhR, AhRV and Arnt in a transformed plant was performed by RT-PCR analysis using a primer which is specific for each gene, after extraction of a whole RNA from a leaf tissue. An actin gene was used as an internal expression index (FIG. 8).

TABLE 2
Screening of transformed tobacco plant (number of
specimens analyzed are shown)

| | | GUS induction with |
|---|---|---|
| | Genomic PCR | AhR ligand treatment |
| | (positive) | (positive) |
| AhR-GUS | 11 individuals | 3 individuals |
| AhRV-GUS | 17 individuals | 8 individuals |

In genomic PCR, integration of three genes (AhR, Arnt, and GUS) was confirmed.

Re-differentiated plants (Kanamycin resistant) were selected.

<Experimental Results>

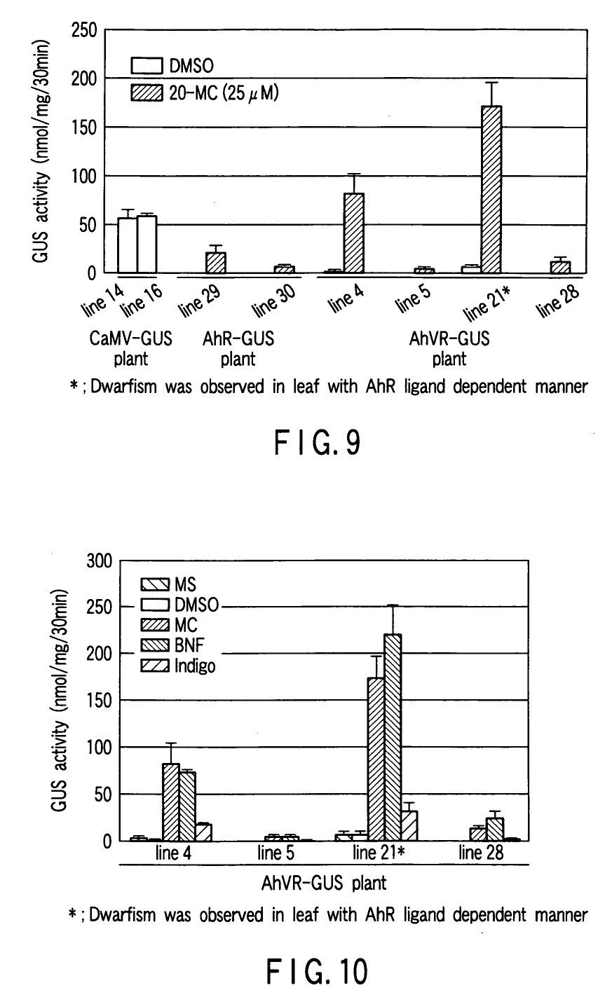

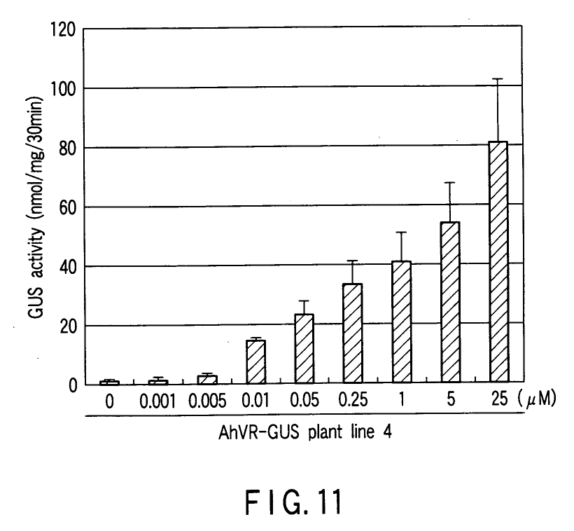

When GUS gene expression analysis using a transformed tobacco plant was performed, an enhanced expression of β-glucuronidase (GUS) was recognized in a plurality of AhR-GUS and AhRV-GUS plants with AhR-ligand treatment dependent manner. Inter alia, the line 4 and line 21 of the AhRV-GUS plant showed a very strong enhancement of the GUS activity dependent on AhR-ligand treatment (20-MC). Also, the lines showed stronger GUS activity compared to an individual which expressed GUS under control of CaMV 35s-P which is widely used as a very strong heterologous gene expression promoter in a plant (FIG. 9).

The similar enhancement of the GUS activity was recognized in the presence of AhR-ligands such as β-NF and indigo, inter alia, the enhancement was remarkable in the presence of 20-MC and β-NF. In addition, when the GUS activities were compared between an MS medium with DMSO (used in a solvent for an AhR-ligand) and an MS medium without addition, a difference was hardly recognized between them (FIG. 10). Further, when AhR-ligand dose dependency was analyzed using 20-MC, the correlation was confirmed between the 20-MC concentration and the GUS activity. And it was confirmed that 5 nM 20-MC in a medium can be detected by the transformed plant (FIG. 1). Expression of GUS was not detected in a control with DMSO (FIG. 12, A). In (A) DMSO treated plant in FIG. 12, expression of GUS was not detected either in leaf (upper figure) or stem (lower figure). However, when cultured on an MS medium containing 20-MC, blue color development (appeared in black in the figure) as a result of remarkable expression of a GUS reporter gene was observed in the tissues (tissues appears on the ground) (FIG. 12, B). In (B) 20-MC-treated plant in FIG. 12, dotted GUS expression was observed over a whole leaf (upper figure). And in a stem (lower figure), strong expression was recognized, in particular, along with fibrovascular bundle.

On the other hand, a whole RNA was extracted from a plant individual which showed enhancement of the specific GUS activity at AhR-ligand treatment. Using the RNA, RT-PCR analysis was performed. The transcription of AhR or AhRV and Arnt into a messenger RNA was confirmed in each individual (FIG. 8). The foregoing results shows that various AhR-ligands are absorbed via a root system of a plant, and that the AhR-ligands activate a mammal AhR-ligand-specific gene expression system at stem and leaf parts.

Therefore, it was demonstrated that a transformed tobacco plant with the present expression system introduced therein can be used as an environment monitoring plant for AhR-ligands such as dioxins.

### Summary of Examples

Example 1 shows an example of a construction for providing a gene expression element XDV/XVD which simplifies a signal transduction system via AhR in a mammal. Originally, AhR forms a heterodimer with Arnt. The heterodimer binds to XRE present in an expression control region of a target gene CYP1A1, to activate transcription of the target gene into an mRNA. However, in XDV/XVD exemplified in Examples, a DNA-binding region was substituted with a DNA-binding region of LexA. Therefore, the XDV/XVD does not need XRE as a binding sequence. The XDV/XVD binds to a LexA promoter sequence as a monomer or as a homodimer. The LexA promoter sequence is a binding sequence of LexA. The XDV/XVD does not require the Arnt for their function.

Since the LexA promoter sequence is a bacterium-derived sequence, pseudo-positive expression by endogenous proteins is very low in a eukaryote used as a host in the present invention. Therefore, since the background can be maintained at low level, it is suitable for the detection of AhR-ligand at an extremely low concentration. Further, a transcriptional activation region of the XDV/XVD is substituted with that of VP16. The transcriptional activation region of VP16 has a very strong transcriptional activity in a yeast, mammal and plant. Therefore, the XDV/XVD has the wide applicability in a eukaryote.

Example 2 shows a transformed plant in which gene expression element of the invention has been introduced. There is provided the technique of on site biomonitoring or reducing pollution in the environment.

References

1. Whitelaw, M., L., et al., The EMBO J. vol. 12 No. 11 pp. 4169-4179, 1993.

2. Whitelock, J., P., Annu. Rev. Pharmacol. Toxicol. 39: pp. 103-125, 1999.

3. Zuo, J., et al., Plant J. 24, pp. 265-273, 2000.

4. Schmidt, J. V., et al., J. Biol. Chem. 268 (29), pp. 22203-22209, 1993.

5. Reisz-Porszasz, S., et al., Molecular and Cellular Biology, 14(9) pp. 6075-6086, 1994.

6. Amy Lusska, et la., J. Biol. Chem, 268(9) pp. 6575-6580, 1993.

7. Sakaki, T., et al., Archives of Biochemistry and Bioohysics, 401 pp. 91-98, 2002

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A vector comprising:
a first promoter sequence which constitutively or conditionally regulates the transcription under control thereof;
a first transcription structure including a DNA-binding region, a nucleus localization signal sequence, an AhR-ligand binding control region and a transcriptional activation region, which is arranged downstream of the first promoter sequence;
one or more second promoter sequence which is specifically coupled with the DNA-binding region, thereby to regulate the transcription under control thereof; and
a second transcription structure including a reporter gene, which is arranged downstream of the second promoter sequence,
wherein the first promoter sequence and the first transcription structure form a first set, the second promoter sequence and the second transcription structure form a second set, and the first and the second sets are arranged in cis- or trans-position on a chromosome or an episome resided within a eukaryotic cell; and
wherein an AhR-ligand which has invaded in the cell binds to a translated product of the first transcription structure, and forms a complex to an amount depending on the AhR-ligand, thereby enhancing transcription of the second transcription structure depending on the amount of the complex formed.

2. The vector according to claim 1, wherein the vector further comprises a third transcription structure including an Arnt gene and/or a drug resistant gene, and said structure is arranged downstream of and transcribed under control of a third promoter sequence, and is further arranged in the same molecule where the first and/or second set are located.

3. The vector according to claim 1, wherein the first and/or third promoter sequence is a CaMV35S promoter or a G-box promoter.

4. The vector according to claim 1, wherein the second promoter sequence is a LexA promoter or an XRE sequence.

5. The vector according to claim 1, wherein the AhR-ligand is dioxins and/or polycyclic aromatic hydrocarbons.

6. The vector according to claim 1, wherein the DNA-binding region is a DNA-binding region of a LexA or a DNA-binding region of AhR.

7. The vector according to claim 1, wherein the nucleus localization signal sequence is derived from SV40.

8. The vector according to claim 1, wherein the transcriptional activation region comprises one or more of transcriptional activation region which derived from AhR or VP16.

9. The vector according to claim 1, wherein the first transcription structure comprises XDV/XVD, AhR or AhRV.

10. The vector according to claim 1, the drug resistant gene included in the third transcription structure is NPTII.

11. The vector according to claim 1, wherein a reporter gene included in the second transcription structure is a gene encoding GUS, GFP or cytochrome p450.

12. A transformant transformed with the vector according to claim 1.

13. The transformant according to claim 12, wherein the transformant is a plant.

14. The transformant according to claim 13, wherein the plant is a higher plant.

15. The transformant according to claim 14, wherein the higher plant is a tobacco plant.

16. The transformant according to claim 12, wherein the transformant is yeast.

17. A transformant, wherein an yeast L40 strain comprising a LexA promoter and a LacZ transcription unit arranged downstream of said promoter in a chromosome has been transformed with a vector comprising XDV/XVD arranged downstream of a GAL1 promoter.

18. A method of monitoring an AhR-ligand, comprising a step of culturing or cultivating the transformant according to any one of the claims 12-17, wherein the AhR-ligand present in the growing environment of the transformant is monitored according to the expression of the reporter gene in the transformant.

19. A method of reducing an AhR-ligand, comprising a step of culturing or cultivating the transformant according to any one of the claims 12-17, wherein the AhR-ligand present in the growing environment of the transformant is metabolized by the reporter gene expressed in the transformant.

20. The method according to claim 18, wherein the AhR-ligand is dioxins and/or polycyclic aromatic hydrocarbons.

21. The method according to claim 19, wherein the AhR-ligand is dioxins and/or polycyclic aromatic hydrocarbons.
